(19) United States
(12) Patent Application Publication
(10) Pub. No.: US 2022/0415482 A1
(43) Pub. Date: Dec. 29, 2022

(54) Systems and Methods for Medical Data Annotation Management

(71) Applicant: Mayo Foundation for Medical Education and Research, Rochester, MN (US)

(72) Inventors: Eric E. Williamson, Rochester, MN (US); Jerome P. Taubel, Rochester, MN (US)

(21) Appl. No.: 17/849,480

(22) Filed: Jun. 24, 2022

Related U.S. Application Data
(60) Provisional application No. 63/215,192, filed on Jun. 25, 2021.

(51) Int. Cl. G16H 30/40; G16H 10/60; G16H 30/20

(52) U.S. Cl. CPC G16H 30/40; G16H 10/60; G16H 30/20

(57) ABSTRACT

The annotation of medical data for use in training an artificial intelligence (“AI”) and/or machine learning (“ML”) algorithm or model is managed to track, validate, and account for compensation of annotation, validation, and adjudication tasks of individual users.

### BACKGROUND

Clinical organizations often have vast archives of medical data, including medical images, clinical data, patient health record data, histopathologic data, and other such medical data. It is often assumed the value associated with these medical data is in the data content itself, but the higher value resides in the physician report that describes the meaning and potential implications of that findings the physician observes in the medical data. The value of archived medical data—particularly medical imaging data—in its native state, therefore, is low in terms of useful value for training artificial intelligence (“AI”) and/or machine learning (“ML”) algorithms or models.

To become valuable, the medical data must be annotated by a human. For instance, organs and lesions in imaging exams must be labeled, segmented, or both. Annotation (e.g., labeling and/or segmentation) informs an AI/ML algorithm or model where to “look” for the information of interest in order to learn patterns or to make other inferences. Medical data annotation is therefore an important aspect to making medical data valuable for training and validating AI/ML algorithms and models.

The expertise of the individual performing annotation work directly impacts the quality of the trained algorithms produced using that data. The most highly skilled individuals are often subspecialty trained physicians. In addition, subspecialty trained physicians who can also correlate different medical data types, such as image annotations and/or segmentations with genomic, proteomic, pathology, lab, or other biologic and EMR metadata produce extremely high value exam data sets. This capability is unique to an integrated medical practice.

Annotation and segmentation are tedious work requiring focused physician time. An example average time for annotating a single imaging exam is approximately 20 minutes. In the routine, day-to-day clinical workflow there is not adequate time available in between cases to markup images. There is a challenge, then, how to incentivize and appropriately compensate for this work, as well as how to manage the annotation of these medical data and track user participation in the annotation of medical data.

### SUMMARY OF THE DISCLOSURE

The present disclosure addresses the aforementioned drawbacks by providing a computer-implemented method for generating and managing annotated medical data using an annotated worklist broker in communication with one or more clients and one or more databases. The method includes retrieving medical data from a database using an annotated worklist broker implemented with a hardware processor and a memory, where the medical data are retrieved in response to a query generated by a first client for a primary annotator. A sample of the medical data is selected with the annotated worklist broker and the sample of the medical data is provided to a second client for a secondary annotator. Primary annotated data are received at the annotated worklist broker from the first client, where the primary annotated data include annotations of the medical data generated by the primary annotator. Secondary annotated data are received at the annotated worklist broker from the second client, where the secondary annotated data include annotations of the sample of the medical data generated by the secondary annotator. The primary annotated data and the secondary annotated data are compared to a verification condition using a computer system, generating output as annotated data. Verified status metadata are attached to the annotated data when the verification condition is satisfied, and unverified status metadata are attached to the annotated data when the verification data is not satisfied. Compensation share metadata are generated and attached or indexed to the annotated data, where the compensation share metadata indicate a respective compensation share attributable at least one of the primary annotator and the secondary annotator.

It is another aspect of the present disclosure to provide a medical data annotation management system that includes a database storing medical data, a first client, a second client, and an annotation worklist broker. The first client is implemented with a hardware processor and a memory and is configured to generate a user interface for annotating medical data received from the database, generating output as first annotated medical data. The second client is implemented with a hardware processor and a memory and is configured to generate a user interface for annotating a sample of medical data received from the database, generating output as second annotated medical data. The annotation worklist broker is implemented with a hardware processor and a memory and is in communication with the database, the first client, and the second client. The annotation worklist broker is configured to generate a work order to retrieve medical data from the database; provide the medical data to the first client; select and provide the sample of medical data to the second client; receive first annotated medical data from the first client; receive second annotated medical data from the second client; compare the first and second annotated medical data, generating output as annotated data; and generate and attach metadata to the annotated data, wherein the metadata indicate a first user of the first client and a first compensation share associated with the first annotated medical data, and a second user of the second client and a second compensation share associated with the second annotated medical data.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a workflow diagram illustrating an example process for annotating and managing medical data.

FIGS. 2A-2C illustrate an example implementation of a compensation model that can be used with the systems and methods described in the present disclosure.

FIG. 3A is a block diagram of an example medical data annotation management system that can implement the methods described in the present disclosure.

FIG. 3B is a block diagram of an example implementation of a portion of the medical data annotation management system of FIG. 3A.

FIG. 4 is a block diagram of an example computer system that can implement aspects of the medical data annotation management systems and methods described in the present disclosure.

### DETAILED DESCRIPTION

Described here are systems and methods for managing the annotation of medical data for use in training an artificial intelligence (“AI”) and/or machine learning (“ML”) algorithm or model.

In general, medical data (e.g., medical images, clinical data) are retrieved from a database for annotation. The medical data are annotated by a primary annotator and the primary annotated data are stored in a database of annotated data. A random sampling of the medical data are also annotated by a secondary annotator and stored as secondary annotated data in the database of annotated data. Metadata associated with the annotators can be attached to the respective annotated data sets, including entity metadata, identifying the annotators; compensation share metadata, identifying a proportional share of royalties or other compensation generated on the annotated data that should be allocated to the respective annotator; and intellectual property (“IP”) share metadata, identifying a proportional share of any IP rights or corresponding royalty shares apportioned based on IP rights that should be allocated to the respective annotator. The primary and secondary annotated data are then processed to verify the accuracy of the annotations. If the annotated data are verified as accurate, they are stored as annotated data. If the annotated data are unverified by failing a condition, then the unverified annotated data are reviewed by an adjudicator. As described below, based on the adjudication process, the compensation shares attributed to the respective annotators can be revised based on whether their annotations are deemed accurate or not by the adjudicator.

The medical data annotation management systems and methods described in the present disclosure can be implemented to annotate previously unannotated medical data (i.e., annotation task); provide additional annotations to already annotated medical data (i.e., reannotation task); validate annotated data, AI/ML algorithms or models trained on annotated data, or both (i.e., validation task), whether in a small scale or a full-scale multi-center clinical trial; and/or verifying annotated medical data through adjudication as necessary (i.e., adjudication task).

In the compensation model(s) described in the present disclosure, an annotator can be allocated a perpetual share in every exam they annotate or segment. If the annotated data they produce are used to train an algorithm that is commercialized, a portion of the revenue returns to the annotator based on metadata in the annotated data that identify the annotator and their proportional compensation share. As multiple products are created, licensed, and sub-licensed by different vendors, the return from these initial annotations can continually be tracked by the metadata created, attached, and managed by the management systems and methods described in the present disclosure.

The model can be extended to include additional steps beyond the initial annotation. For instance, annotation can be performed by a second expert annotator as part of a quality assurance (“QA”) or validation step, as mentioned above. This step could be used in a supervisory capacity to validate results from a less experienced annotator. Such a process could be helpful as part of an “on boarding” process for new investigators, including external partners.

In some cases, the second expert can perform this QA step in a blinded fashion. This blinded review will allow the determination of inter-observer variability. Thresholds can be set for agreement between the primary and secondary annotators to ensure accurate data curation. If the preset threshold for inter-observer agreement are met, the case can be marked as verified (or concordant) and no further annotations would be necessary. If the preset threshold is not met, the case would be marked unverified (or discordant) and a third expert annotator would be employed to adjudicate the results of the case. This adjudicator role would be compensated as a part of the sharing model and the discordant annotator would receive a diminished share, as described below in more detail.

As mentioned, a similar system can be used to validate the results from individual AI/ML algorithms. Once an algorithm has been trained, its output is validated under clinical use conditions before being deployed into practice. This validation should be performed by an expert who can accept, reject, or edit the AI results. In such a case, the share of the validator can be attached as compensation share metadata to the medical data, images, feature maps, or other output data produced by the algorithm, rather than to the input medical data. The validator's share will be tracked and managed similar to the shares of the initial annotator(s) and adjudicator.

The annotation management systems and methods can be further extended to compensate for the use of a vendor's or other stakeholder's proprietary discovery toolset or raw data set used to create medical data and/or annotated data. In such instances, the vendor or stakeholder can be assigned a proportional compensation share, stored in metadata attached or indexed to the annotated medical data.

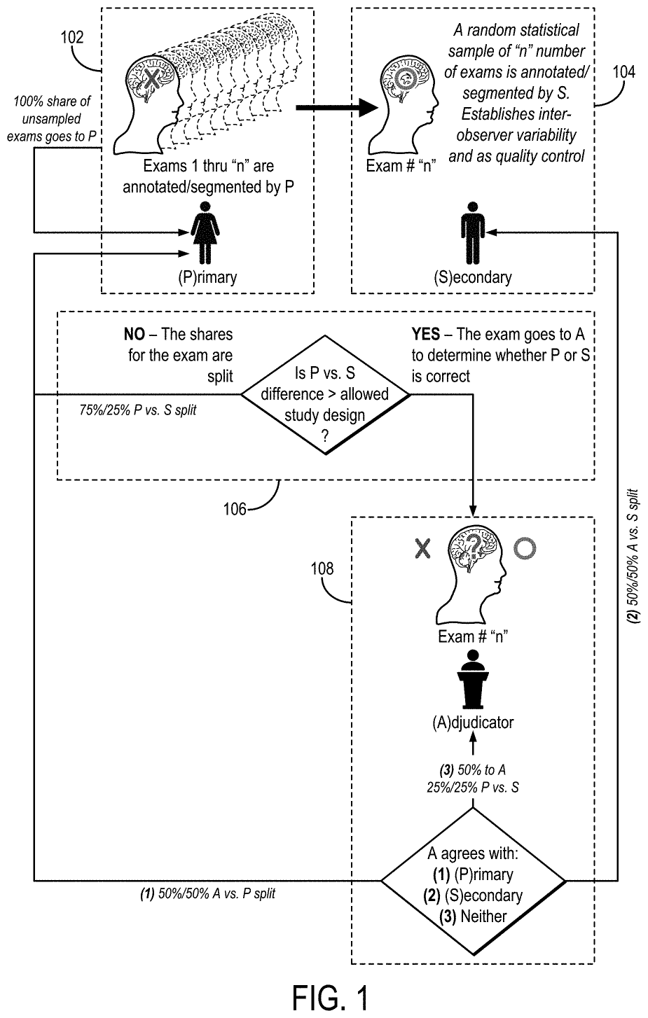

Referring now to FIG. 1, a workflow diagram is illustrated as setting forth the general process 100 for implementing a medical data annotation management system to annotate, validate, and/or adjudicate medical data for use in an AI/ML algorithm or model, such as to train the AI/ML algorithm or model.

Medical data (e.g., medical images, as shown in FIG. 1) are provided for annotation, which may include retrieving the data from a database in response to a work order, or by selecting data from a worklist. The medical data can be provided to a computer system by retrieving medical data from a database, a memory, or another data storage device or medium. For instance, the computer system can initiate a request to the database, memory, or other data storage device or medium to retrieve one or more medical data sets for annotation. Additionally or alternatively, the computer system can generate and provide a list of medical data that have been added to a worklist for annotation. In this way, the user can retrieve one or more medical data sets from the list of studies that require annotation.

A primary annotator reviews and annotates the medical data, generating output as primary annotated data, as indicated at step 102 of the example process 100 shown in FIG. 1. A random sample of the medical data are also provided to a different user who is a secondary annotator. The secondary annotator also annotates the same medical data sets selected in the random sample, generating output as secondary annotated data, as indicated at step 104 of the example process 100 shown in FIG. 1. As one example, the size of the random statistical sample can be selected or otherwise determined based on a study design. For a small or difficult study design, the sample size can be larger, such as upwards of 100% of the medical data. As another example, the size of the random statistical sample can be selected without consideration of any particular study design. In these instances, the sample size can be selected based on the overall size of the provided medical data (e.g., to maintain a level of statistical significance relative to the number of medical data sets), or arbitrarily.

In general, annotating the medical data includes adding metadata to the medical data sets. The annotations can include semantic annotations, image annotations, entity annotations, or other suitable data annotation types. The added metadata can include tags or labels to text data, image data, video data, or so on.

As a non-limiting example, the annotations can be image annotations, which can include bounding boxes (e.g., 2D bounding boxes, 3D bounding boxes), polygons, lines, splines, semantic segmentations, and/or combinations thereof. In general, image annotations can be used to segment and label different anatomical and/or physiological features in medical image data. For instance, image annotations can include semantic segmentations that segment different tissues in a medical image. As another example, image annotations can include lines or splines that demarcate and/or measure the dimensions of features or distances between features in a medical image. Any suitable image annotation can be recorded and stored with the medical data.

The medical data annotated by the primary annotator is tagged with primary annotator metadata. The primary annotator metadata can include entity metadata, such as the name of the primary annotator, the organization employing the primary annotator, the business unit or department to which the primary annotator belongs, or the like. The primary annotator metadata can also include compensation metadata, which indicates a proportional share of royalties that should be paid to the primary annotator as compensation for the annotation task, which is described below in more detail. Each time the primary annotated medical data are retrieved for use, the primary annotator metadata can be read to identify that compensation should be allocated for the primary annotator according to the proportional share recorded in the compensation metadata. In some instances, the primary annotator metadata can also include intellectual property (“IP”) metadata indicating a proportional share of any IP rights that the primary annotator may have in the annotated medical data.

Similarly, the medical data annotated by the secondary annotator is tagged with secondary annotator metadata. The secondary annotator metadata can include entity metadata, compensation metadata, and/or IP metadata, as discussed above.

When a medical data set that has been annotated by the primary and/or secondary annotator is stored as a verified annotated data set, the respective annotator profile in tagged with metadata that indicates the successful completion of the annotation task. For instance, the annotator profile can be updated with metadata that includes an increment to the number of successful annotation tasks completed by the annotator. The number of successful annotation tasks can be used by management system to promote the annotator, as described below.

As a non-limiting example, when the medical data include medical image data, the primary annotator metadata can be stored in a DICOM header of the annotated medical images or in a separate database indexed to the annotated images. For instance, the primary annotator metadata can be stored at the DICOM exam series level of the DICOM header's hierarchy.

The primary and secondary annotated data are then compared to verify whether a particular threshold or study design condition has been satisfied with the annotation tasks performed by the primary and secondary annotators, as indicated at step 106 of the example process 100 shown in FIG. 1. For example, a percent difference between the primary and secondary annotated data can be computed and compared against a condition for verification, such as a threshold. If the comparison results in the condition being satisfied, the primary and/or secondary annotated data are stored in a database as verified annotated data.

Compensation shares are assigned to the primary and secondary annotators and attached, or otherwise indexed, to the annotated data as metadata according to a stored compensation model. In the example where the annotated data include annotated medical images, the compensation shares can be attached as metadata stored in the DICOM header of the images, which may be stored at the exam series level, or in a separate databased indexed to the annotated images.

In general, the compensation model can be structured to incentivize and reward user participation in the creation of a valuable annotated and curated data repository.

As a non-limiting example, the primary annotator can be assigned a 75% share and the secondary annotator can be assigned a 25% share, such that the primary annotator is awarded a 75% share of any royalties generated on the use of the annotated data, and the secondary annotator is awarded a 25% share of any royalties generated on the use of the annotated data.

Shares can be determined on a case-by-case basis or can be set by policy, such as an institutional policy, department policy, or other policy. Additionally, if a vendor or other stakeholder has a stake due to the use of proprietary resources in the collection and/or annotation of the medical data, a compensation share can be assigned to that vendor and/or stakeholder.

If the comparison results in the condition being unsatisfied, the annotated data are flagged as being unverified. The unverified annotated data are then reviewed by an adjudicator, as indicated at step 108. For instance, unverified annotated data can be pushed to a worklist queue for an adjudicator, or an adjudicator can query a database to retrieve unverified annotated data that need to be reviewed for further verification. The adjudicator can review and verify the annotated data by performing an annotation of the original underlying medical data, or by comparing the annotations in the primary and secondary annotated data.

Based on the adjudicator's review of the unverified annotated data, which may be blinded and anonymous, the compensation share attached as metadata, or otherwise indexed, to the annotated data can be updated. For instance, when the adjudicator determines that the primary annotator's annotation is correct, then the primary annotated data are stored as the annotated data and the compensation share can be reapportioned between the primary annotator and the adjudicator. As an example, the primary annotator can be reassigned a 50% share and the adjudicator can be assigned a 50% share.

When the adjudicator determines that the secondary annotator's annotation is correct, then the secondary annotated data are stored as the annotated data and the compensation share can be reapportioned between the secondary annotator and the adjudicator. As an example, the secondary annotator can be reassigned a 50% share and the adjudicator can be assigned a 50% share.

When the adjudicator determines that neither the primary nor the secondary annotator's annotations are correct, then the adjudicator's annotation of the medical data is stored as the annotated data and the compensation share can be reapportioned between the primary annotator, the secondary annotator, and the adjudicator. As an example, the primary annotator can be reassigned a 25% share, the secondary annotator can be reassigned a 25% share, and the adjudicator can be assigned a 50% share.

The medical data annotation management workflow can account for promotion and demotion of individuals who are identified as primary annotator, secondary annotation, and adjudicator. For instance, if a primary annotator's annotations are regularly rejected by an adjudicator, then the primary annotator can be demoted in the management system to a secondary annotator. Likewise, if a secondary annotator's annotations are regularly rejected by an adjudicator, then the secondary annotator can be removed from the management system as an authorized annotator. In either case, the management system can also flag the demoted annotator for further training or retraining on the annotation tasks. Conversely, a secondary annotator whose annotations are regularly verified can be promoted to a primary annotator, and a primary annotator whose annotation are regularly verified can be promoted to an adjudicator in the management system. In this way, the management system can provide a quality control of the annotators and, as a result, the annotated data being created.

When an adjudicator routinely sides with one annotator over others, the adjudicator's work product can be flagged for review and verification, such as by a different authorized adjudicator. If the offending adjudicator is determined to be improperly siding with one annotator over others, then the offending adjudicator can be excluded as an authorized adjudicator from the management system, flagged for retraining, or both. Similarly, when an adjudicator routinely disagrees with both primary and secondary annotators, the management system can flag the adjudicator's work for independent review by a different authorized adjudicator. Additionally or alternatively, the management system can generate and attach, or otherwise index, metadata to the annotated data that indicates that the study design should be reviewed. For instance, the inter-observer agreement threshold used for the study design can be changed in response to the failure rate of the annotated data. The management system can also be configured to generate an alert message indicating that the study design should be reviewed.

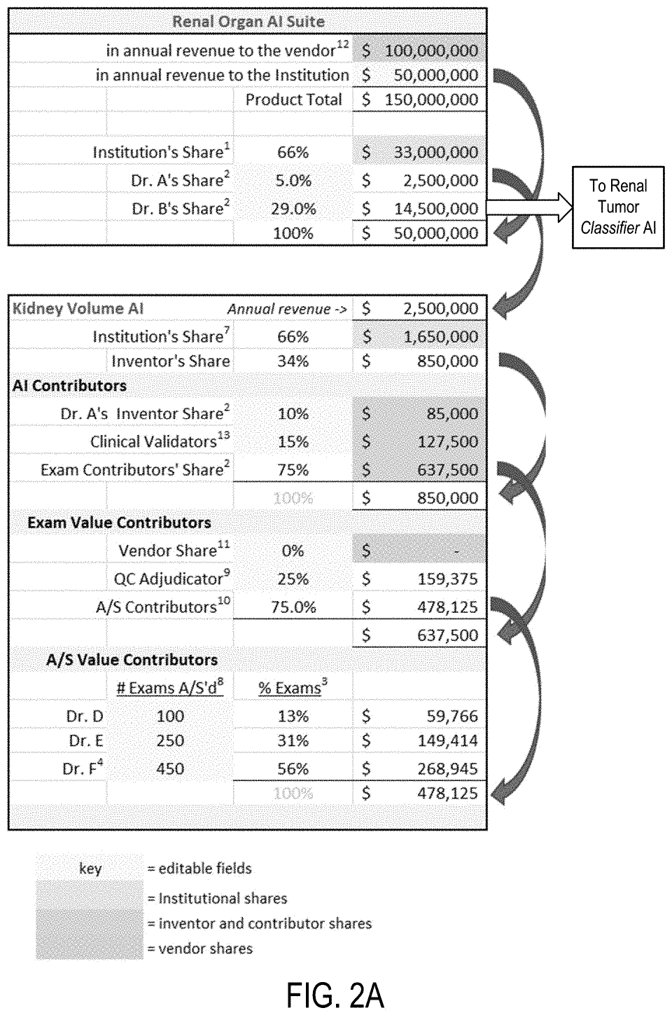

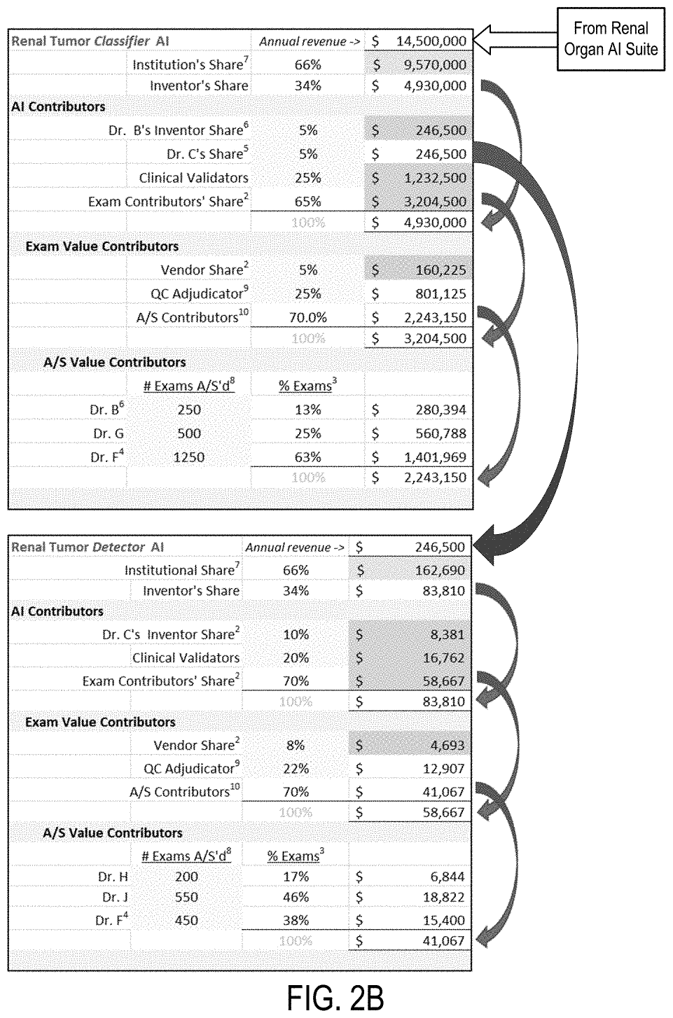

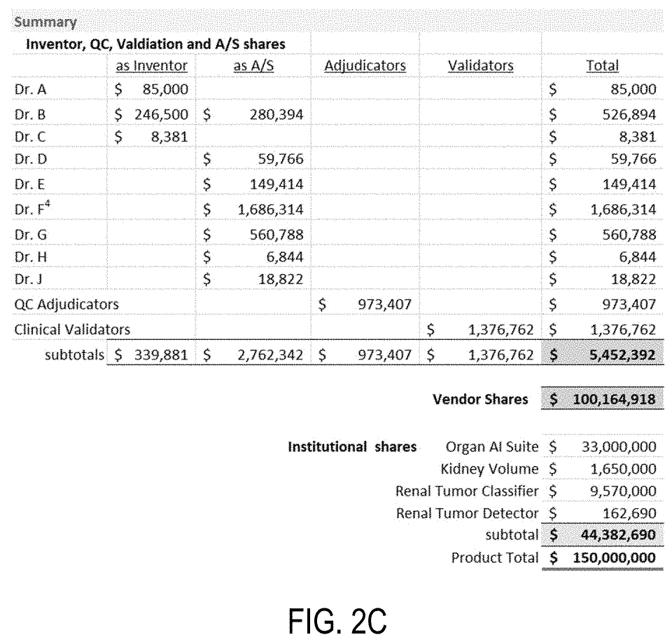

An example implementation of a compensation model that can be used with the systems and methods described in the present disclosure is illustrated in FIGS. 2A-2C. The footnotes in this example implementation of a compensation model are as follows. Footnote #1 indicates that the institutional share values can be set by the company and institution/physician. Footnote #2 indicates that exam contributors can be any stakeholder who contributed value when preparing the exam level data for use in training an AI algorithm or model. Shares in this example model can be determined case-by-case, or can be set by standing policy or agreement. If a vendor has a stake due to use of proprietary resources, they could be granted a share. Multiple vendors tools may be involved in complex projects and the share negotiated for each. The vendor share can alternatively be declared at the AI contributor level or the annotator/segmenter (“A/S”) contributor level. As a non-limiting example, a 10/90 inventor versus A/S contributor split can be used.

Footnote #3 indicates that shares are split proportionally among A/S team based on % individual exam contribution in this example. Footnote #4 indicates that, in this example, Dr. F's annotated data are used in all three projects. Two projects use the exact same exams. Dr. F therefore appropriately benefits from their effort multiple times in multiple ways. Footnote #5 indicates that, in this example, in order to function, the classifier AI requires the detector AI as a base. Therefore, a portion of the royalty revenue flows back to the detector AI team. This royalty flow can be managed by tracking the compensation share metadata attached, or otherwise indexed, to the annotated data. Additionally or alternatively, compensation share metadata can be attached, or otherwise indexed to, the trained detector AI algorithm or model, such that when the AI algorithm or model is used the appropriate compensation shares are allocated to the A/S team.

Footnote #6 indicates that Dr. B is both the AI inventor and an A/S contributor, and is allocated different inventor compensation shares and annotator compensation shares, as discussed above. Footnote #7 indicates that the institutional share in this example includes a department share. The department share can be broken out as a separate variable if the department wanted to contribute a part of the department share to the inventors or the A/S team. Footnote #8 indicates that the annotated/segmented exams can be placed in an indexed, curated data repository that tracks the A/S contributors' effort for each exam. The repository can be available to the institution or the inventor to create products, services, or new revenue streams. This may be located within a longitudinal patient record, vendor neutral archive, a results delivery platform, with a Cloud partner, or so on.

Footnote #9 indicates that QC/QA adjudicator roles can perform a quality check on A/S exams to insure they fall within the acceptance criteria for inclusion in a given AI algorithm project. The share percentage may be set by policy. Adjudicators may also be A/S contributors. Footnote #10 indicates that A/S contributors are individuals who perform the annotation, segmentation, and/or data preparation work that provides the inputs to training the AI. Footnote #11 indicates that, in this kidney volume AI example, no proprietary vendor software was used, so there is a 0% vendor share. Footnote #12 indicates that, in this example, the compensation model implements a 33% versus 67% vendor versus institution revenue split. Footnote #13 indicates that clinical validators are individual physicians who confirm that the output of the trained AI algorithm produces clinically valid results. The share splitting can be the same splitting model as described for the A/S contributors.

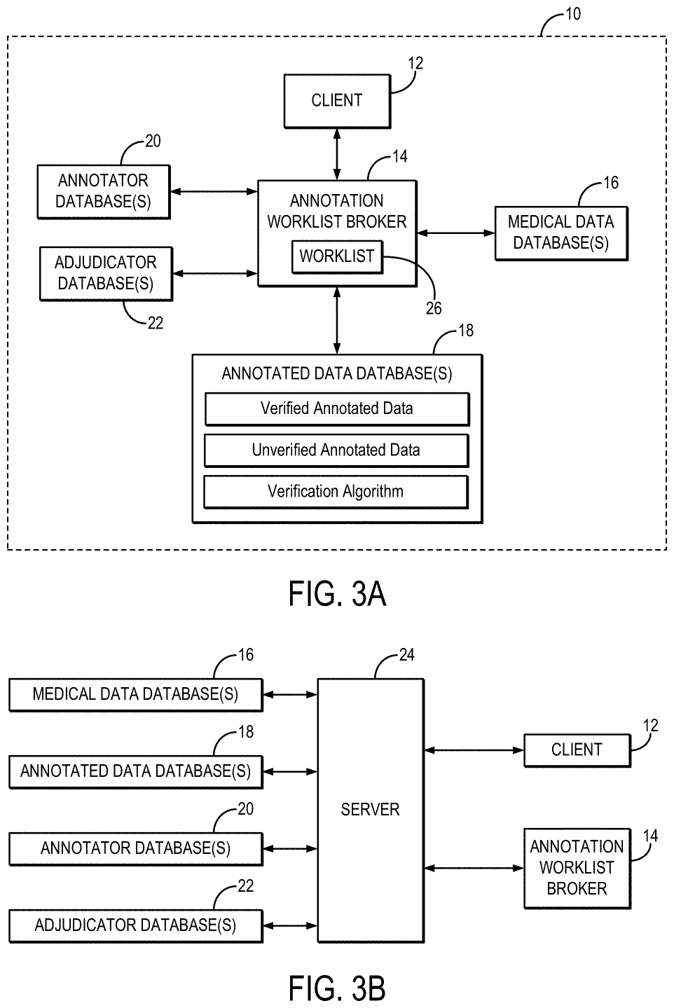

FIG. 3A illustrates an example medical data annotation management system 10. The system 10 includes a client 12 that communicates with an annotation worklist broker 14 to order annotation of medical data, validation of annotated medical data, and/or adjudication of annotated medical data depending on the user and desired task. The annotation worklist broker 14 is in communication with several databases, including one or more medical data database(s) 16, annotated data database(s) 18, annotator database(s) 20, and adjudicator database(s) 22.

In general, the medical data database(s) 16 store unannotated medical data, such as medical image data, clinical data, patient record data, histopathology data, or other such medical data.

The annotated data database(s) 18 store annotated medical data, including annotated medical data that have been verified as satisfactory (i.e., verified annotated data) by consensus of a primary annotator, a secondary annotator, and/or an adjudicator as outlined below. The annotated data database(s) 18 can also store annotated medical data that are stored in an unverified state (i.e., unverified annotated data), such as when medical data that have been annotated by a primary annotator and a secondary annotator do not satisfy a comparison or validation condition. Such unverified annotated data can be stored in the annotated data database(s) 18 until acted upon by an adjudicator or returned to the medical data database(s) 16 for annotation by different primary and/or secondary annotators.

The annotator database(s) 20 store profiles and associated metadata for various authorized annotators. For example, an annotator profile can include data and associated metadata, such as a user ID, an organization or department ID, the annotator level (i.e., primary or secondary), the number of successfully completed annotations, the percentage of verified annotations relative to unverified annotations completed by the annotator, the percentage of unverified annotations resolved in favor of the annotator (e.g., a success rate), the percentage of unverified annotations not resolved in favor of the annotator (e.g., a failure rate), a list of unique IDs (“UIDs”) for verified annotated data completed by the annotator, and other such data or metadata pertinent to the annotator and their completion of annotation and validation tasks.

The adjudicator database(s) 22 store profiles and associated metadata for various authorized adjudicators. For example, an adjudicator profile can include data and associated metadata, such as a user ID, an organization or department ID, their subject matter area(s) of competence and/or expertise, the number of successfully completed adjudications, the percentage of unverified annotations resolved by the adjudicator (e.g., a success rate), the percentage of unverified annotations unable to be resolved by the adjudicator (e.g., a failure rate), a list of unique IDs (“UIDs”) for verified annotated data successfully adjudicated by the adjudicator, and other such data or metadata pertinent to the adjudicator and their completion of adjudication tasks.

As shown in FIG. 3B, communication between the client 12, the annotation worklist broker 14, and the databases (e.g., medical data database(s) 16, annotated data database(s) 18, annotator database(s) 20, adjudicator database(s) 22) can be implemented via a server 24 that is configured to operate as a service layer or middleware. The annotation worklist broker 14 may implement, for example, HL7 standards, DICOM standards, or other suitable standards for various types of medical data.

The client 12 can include a hardware processor, a memory, one or more inputs, and a display. In some examples, the client 12 can include a desktop computer, a laptop computer, a tablet device, a mobile device, or the like. The databases can be any suitable database for storing information such as medical data, including medical images, and associated metadata (e.g., medical data database(s) 16); annotated data and associated metadata (e.g., annotated data database(s) 18); annotator profiles and associated metadata (e.g., primary and/or secondary annotator database(s) 20); and/or adjudicator profiles and associated metadata (e.g., adjudicator database(s) 22). In some examples, the database(s) can implement a SQL database. In some instances, such as when the medical data include medical images, one or more of the databases may be implemented as an archive, such as a Picture Archiving and Communications System (“PACS”), a vendor neutral archive (“VNA”), a long term archive (“LTA”), or other suitable archive for storing medical images and associated metadata on a short-term or long-term basis.

The annotation worklist broker 14 can generally include a hardware processor and a memory. In some implementations, the worklist broker 14 can receive orders from the client 12 via an HL7 standard, a DICOM standard, or other suitable standard, to retrieve medical data from the medical data database(s) 16 for annotation (i.e., an annotation work request). In these implementations, the annotation worklist broker 14 can fetch unannotated medical data from the medical data database(s) 16 according to parameters (e.g., data type, anatomy type, patient type) submitted or otherwise queried by the user. In similar implementations, the worklist broker 14 can receive orders from the client 12 via an HL7 standard, a DICOM standard, or other suitable standard, to retrieve annotated data from the annotated data database(s) 16 for validation (i.e., a validation work request) or adjudication (i.e., an adjudication work request), or additional annotation (e.g., adding labels to previously segmented images). In these implementations, the annotation worklist broker 14 can fetch annotated data (e.g., verified or unverified annotated data) from the annotated data database(s) 18 according to parameters (e.g., data type, anatomy type, patient type) submitted or otherwise queried by the user.

In other implementations, the annotation worklist broker 14 can locally store a worklist 26 identifying various work tasks (e.g., annotation, validation, adjudication) that are pending or otherwise queued for medical and/or annotated data. In these implementations, a user can query the worklist 26 and select one or more medical and/or annotated data sets that have been queued for different work tasks. For instance, if the user is a primary or secondary annotator, the user can select medical data sets from the worklist 26 that have been queued for annotation. The annotation worklist broker 14 will then retrieve the corresponding medical data from the medical data database(s) 16 for annotation in response to the user's selection. Additionally or alternatively, a primary and/or secondary annotator can select annotated data sets from the worklist that have been queued for validation. The annotation worklist broker 14 will then retrieve the corresponding annotated data from the annotated data database(s) 18 for validation in response to the user's selection. Similarly, if the user is an adjudicator, the user can select unverified annotated data from the worklist 26 that have been queued or otherwise flagged for adjudication. The annotation worklist broker 14 will then retrieve the corresponding annotated data from the annotated data database(s) 18 for adjudication in response to the user's selection.

The client 12 generally provides a user interface through which a user can communicate requests to the annotation worklist broker 14. For instance, a user can generate a work order (e.g., an annotation work order, a validation work order, an adjudication work order) for annotating medical data and/or validating or adjudicating annotated data at the client 12 and this work order can be processed by the annotation worklist broker 14 to query the respective database(s) and retrieve the selected medical and/or annotated data.

To this end, a work order can include study metadata input by the user at the client 12. Study metadata may include metadata information about the patient, the data type (e.g., medical image data, clinical data), the organization that owns the data, and so on.

As described, the annotation worklist broker 14 can also create and attach metadata to annotated data. For instance, the annotation worklist broker 14 can create entity metadata, compensation share metadata, IP share metadata, and other such metadata. These metadata can then be attached to the annotated data created by the user. The annotated data are thus sent to the annotated data database(s) 18 with these additional metadata being attached thereto. Additionally or alternatively, the annotation worklist broker 14 can create these metadata and index them to the annotated data. In these instances, the metadata can be stored in a separate database indexed to the annotated data.

The annotated data database(s) 18 implement an algorithm to verify annotated data received from the annotation worklist broker 14. The verification algorithm operates on metadata contained in the annotated data (e.g., the DICOM header for medical images, or other metadata) to identify whether the annotated data were created by a primary or secondary annotator, and to verify the accuracy of the annotation by comparing annotated data generated from the same input medical data by different primary and secondary annotators.

The medical data annotation management system 10 described here can implement a thin client application on the client 12 and that works together with the annotation worklist broker 14 and databases (e.g., medical data database(s) 16, annotated data database(s) 18, annotator database(s) 20, adjudicator database(s) 22) to create, manage, validate, adjudicate and/or store annotated medical data and related information. As such, the described system 10 can securely store and make accessible medical data for annotation; track which individuals performed annotation, validation, and/or adjudication tasks; apportion and track compensation levels for different individuals; and manage the promotion and/or demotion of individuals between job roles, such as primary annotator, secondary annotator, and adjudicator.

Users can launch an application at the client 12 (e.g., the client application) to both place a new work order and view any outstanding work order requests (e.g., to annotate, validate, verify, and/or adjudicate data). Additional views provided on the user interface of the client 12 can include a historical search for viewing and an ability to edit or cancel past work order entries stored in the worklist 26 that are not in a completed state.

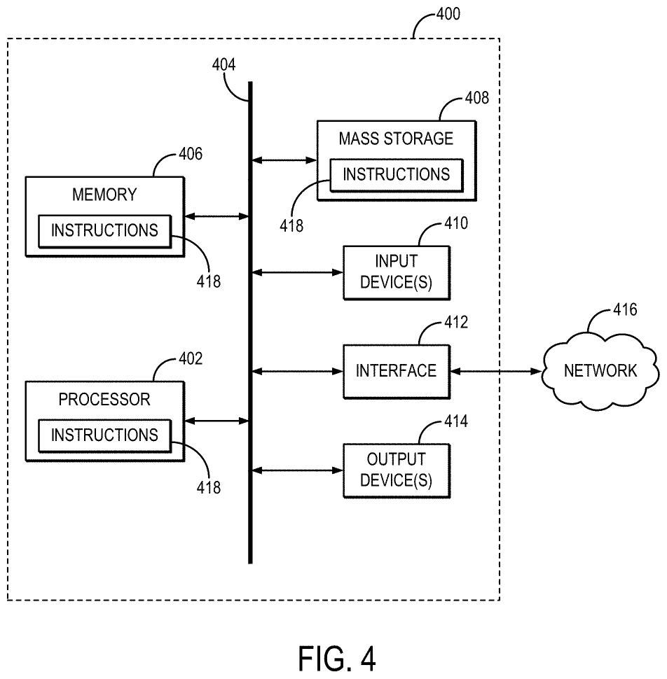

FIG. 4 is a block diagram illustrating an example of a computer system 400 that can implement systems, methods, and algorithms described here. The computer system 400 can include a processor 402 that is coupled to an interconnect 404, which may be an interconnection bus or the like. As an example, the processor 402 can be any suitable processor, processing unit, or microprocessor. Furthermore, the processor 402 may include a single processor or multiple different processors that are coupled to the interconnect 404.

The processor 402 is coupled to a memory 406 via the interconnect 404. The memory 406 can include any type of volatile memory, non-volatile memory, or combinations of both, including static random access memory (“SRAM”), dynamic random access memory (“DRAM”), flash memory, read-only memory (“ROM”), and so on.

The computer system 400 also includes a mass storage device 408, one or more input devices 410, an interface 412, and one or more output devices 414 that are connected to the interconnect 404. The one or more input devices 410 may include a keyboard, a mouse, a touch screen display, and so on. The interface 412 may be any suitable interface for wired or wireless communication between the computer system 400 and another computer system via a network 416. The one or more output devices 414 may include a display or the like.

The mass storage device 408 can include a machine-readable medium on which is stored one or more sets of data structures and instructions 418 (e.g., software) embodying or utilized by any one or more of the systems, methods, or algorithms described here. The instructions 418 may also reside, completely or at least partially, within the memory 406 or a local memory within the processor 402. The instructions 418 may also be transmitted or received over the network 416 and received by the computer system 400 via the interface 412.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A method for generating and managing annotated medical data using an annotated worklist broker in communication with one or more clients and one or more databases, the method comprising:
(a) retrieving medical data from a database using an annotated worklist broker implemented with a hardware processor and a memory, wherein the medical data are retrieved in response to a query generated by a first client for a primary annotator;
(b) selecting a sample of the medical data with the annotated worklist broker and providing the sample of the medical data to a second client for a secondary annotator;
(c) receiving, at the annotated worklist broker, primary annotated data from the first client, wherein the primary annotated data comprise annotations of the medical data generated by the primary annotator;
(d) receiving, at the annotated worklist broker, secondary annotated data from the second client, wherein the secondary annotated data comprise annotations of the sample of the medical data generated by the secondary annotator;
(e) comparing the primary annotated data and the secondary annotated data to a verification condition using a computer system, generating output as annotated data and attaching verified status metadata to the annotated data when the verification condition is satisfied and unverified status metadata to the annotated data when the verification data is not satisfied; and
(f) generating and attaching to the annotated data, with the computer system, compensation share metadata indicating a respective compensation share attributable at least one of the primary annotator and the secondary annotator.

2. The method of claim 1, wherein the compensation share metadata is generated according to a compensation model.

3. The method of claim 2, wherein the compensation model indicates a first compensation share to the primary annotator and a second compensation share to the secondary annotator, wherein the first compensation share is greater than the second compensation share.

4. The method of claim 1, further comprising:
providing annotated data having attached thereto unverified status metadata to a third client for an adjudicator;
receiving, at the annotated worklist broker, third annotated data from the third client, wherein the third annotated data comprise annotations of the medical data generated by the adjudicator; and
updating the compensation share metadata in response to the third annotated data.

5. The method of claim 4, wherein the compensation share metadata are updated to allocate a first compensation share to the primary annotator and a second compensation share to the adjudicator when the third annotated data indicate that the second annotated data failed the verification condition.

6. The method of claim 4, wherein the compensation share metadata are updated to allocate a first compensation share to the secondary annotator and a second compensation share to the adjudicator when the third annotated data indicate that the first annotated data failed the verification condition.

7. The method of claim 4, wherein the compensation share metadata are updated to allocate a first compensation share to the primary annotator, a second compensation share to the secondary annotator, and a third compensation share to the adjudicator when the third annotated data indicate that both the first annotated data and second annotated data failed the verification condition.

8. The method of claim 1, wherein the medical data comprise medical image data.

9. The method of claim 8, wherein the annotated data comprise at least one of labeled medical image data and segmented medical image data.

10. The method of claim 8, wherein the compensation share metadata are attached to the medical image data as an exam series level of a DICOM header in each image of the medical image data.

11. A medical data annotation management system, comprising:
a database storing medical data;
a first client implemented with a hardware processor and a memory, the first client being configured to generate a user interface for annotating medical data received from the database, generating output as first annotated medical data;
a second client implemented with a hardware processor and a memory, the second client being configured to generate a user interface for annotating a sample of medical data received from the database, generating output as second annotated medical data;
an annotation worklist broker implemented with a hardware processor and a memory and in communication with the database, the first client, and the second client, the annotation worklist broker being configured to:
generate a work order to retrieve medical data from the database;
provide the medical data to the first client;
select and provide the sample of medical data to the second client;
receive first annotated medical data from the first client;
receive second annotated medical data from the second client;
compare the first and second annotated medical data, generating output as annotated data; and
generate and at least one of attach or index metadata to the annotated data, wherein the metadata indicate a first user of the first client and a first compensation share associated with the first annotated medical data, and a second user of the second client and a second compensation share associated with the second annotated medical data.

12. The system of claim 11, wherein the annotation worklist broker is configured to access a compensation model from a memory and to calculate the first compensation share and the second compensation share according to the compensation model.

13. The system of claim 11, wherein the annotation worklist broker is configured to compare the first and second annotated medical data relative to a verification condition; and to set an unverified status flag in the metadata as true if the verification condition is not satisfied.

14. The system of claim 13, further comprising a third client implemented with a hardware processor and a memory, the third client being configured to generate a user interface for adjudicating the annotated data, generating output as adjudicated annotated data; and wherein the annotation worklist broker is configured to update the metadata and to at least one of attach or index the updated metadata to the adjudicated annotated data.

15. The system of claim 14, wherein the annotation worklist broker is configured to calculate a third compensation share based on the adjudicated annotated data.

16. The system of claim 14, wherein the annotation worklist broker is configured to update the metadata by allocating the first compensation share to the first user and the second compensation share to a third user of the third client when the adjudicated annotated data indicate that the second annotated medical data failed the verification condition.

17. The system of claim 14, wherein the annotation worklist broker is configured to update the metadata by allocating the first compensation share to the second user and the second compensation share to a third user of the third client when the adjudicated annotated data indicate that the first annotated medical data failed the verification condition.

18. The system of claim 14, wherein the annotation worklist broker is configured to update the metadata by allocating the first compensation share to the first user, the second compensation share to the second user, and a third compensation share to a third user of the third client when the adjudicated annotated data indicate that both the first annotated data and second annotated data failed the verification condition.

19. The system of claim 11, wherein the annotated data comprise annotated medical image data and the annotation worklist broker is configured to attach or index the metadata to the annotated data as an exam series level of a DICOM header in each image of the annotated medical image data.
